(19) United States
(12) Patent Application Publication
(10) Pub. No.: US 2018/0196022 A1
(43) Pub. Date: Jul. 12, 2018

(54) GAS SENSOR

(71) Applicant: Integrated Device Technology, Inc., San Jose, CA (US)

(72) Inventors: Srikanth KULKARNI, San Jose, CA (US); Viresh PATEL, San Jose, CA (US); Jitesh SHAH, San Jose, CA (US); George DELTORO, San Jose, CA (US)

(21) Appl. No.: 15/405,089

(22) Filed: Jan. 12, 2017

(51) Int. Cl. G01N 33/00

(52) U.S. Cl. CPC G01N 33/0016; G01N 33/0027

(57) ABSTRACT

In accordance with some embodiments of the present invention, a gas sensor system is disclosed. In accordance with some embodiments, a system includes a glass substrate; a heater formed on the glass substrate; and a sensor formed adjacent the heater formed on the glass substrate. A method of forming a gas sensor system according to some embodiments includes providing a glass substrate; forming a heater on the glass substrate; and forming a sensor adjacent the heater on the glass substrate.

### TECHNICAL FIELD

Embodiments of the present invention are related to gas sensors.

### DISCUSSION OF RELATED ART

Gas sensors find uses in a wide variety of domestic, commercial and industrial applications. Gas sensors can be formed on semiconductor chips for ease of manufacture. However, some problems with the manufacture and operation of gas sensors on silicon substrates have been detected. In particular, in order to have high efficiency operation of the gas sensor, the gas sensor should be held at an elevated temperature, which may put damaging thermal stress on the sensor and the substrate and may require high energy usage.

Therefore, there is a need to develop better gas sensors and processes for forming gas sensors.

### SUMMARY

In accordance with some embodiments of the present invention, a gas sensor system is disclosed. In accordance with some embodiments, a system includes a glass substrate; a heater formed on the glass substrate; and a sensor formed adjacent the heater formed on the glass substrate. A method of forming a gas sensor system according to some embodiments includes providing a glass substrate; forming a heater on the glass substrate; and forming a sensor adjacent the heater on the glass substrate.

These and other embodiments are further discussed below with respect to the following figures.

### BRIEF DESCRIPTION OF THE DRAWINGS

FIG. 1 illustrates a conventional gas sensor system.

FIGS. 2A-2C illustrate formation of the conventional gas sensor system illustrated in FIG. 1.

FIG. 3 illustrates a gas sensor system according to embodiments of the present invention.

FIGS. 4A through 4C illustrate an example method of forming the gas sensor system illustrated in FIG. 3.

FIG. 5 illustrates another gas sensor system according to some embodiments of the present invention.

FIGS. 6A through 6C illustrate an example method of forming the gas sensor system illustrated in FIG. 5.

FIGS. 7A through 7D illustrate embedding a gas sensor system as illustrated in FIGS. 3 and 5 into a system.

### DETAILED DESCRIPTION

In the following description, specific details are set forth describing some embodiments of the present invention. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure.

This description and the accompanying drawings that illustrate inventive aspects and embodiments should not be taken as limiting—the claims define the protected invention. Various changes may be made without departing from the spirit and scope of this description and the claims. In some instances, well-known structures and techniques have not been shown or described in detail in order not to obscure the invention.

Elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

The sensitivity of the gas sensors such as the ones developed using Metal Oxide (MOX) improves when operating at elevated temperatures. The optimum temperature for sensing may vary based on the sensor material used, the gas to be detected and the product design. However, typical optimal temperatures may be in the range between 200° C. and 500° C. The optimal temperature can be achieved and maintained by depositing the sensor on a substrate that can be heated. In addition to the sensitivity of the sensor, the electric power required to heat the sensor to a desired temperature and maintain that temperature for the required duration is a critical device parameter.

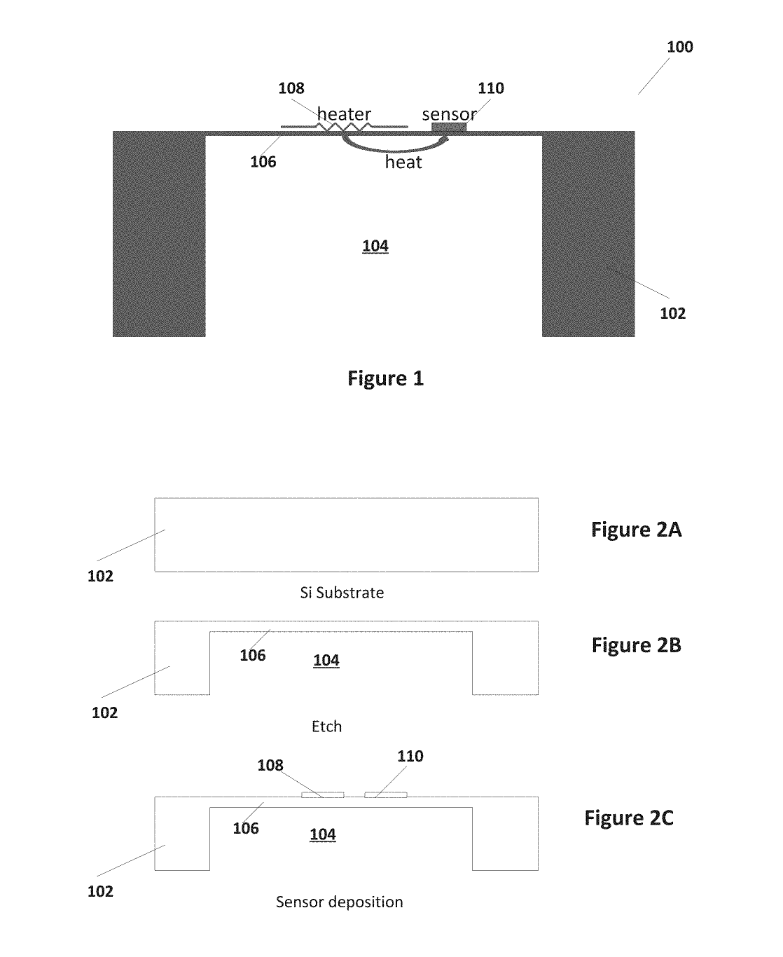

FIG. 1 illustrates a conventional gas sensor system 100. As shown in FIG. 1, gas sensor system 100 includes a gas sensor 110 mounted on a silicon wafer 102. A heater 108 is mounted adjacent to gas sensor 110 in order to heat sensor 110 to a desired temperature. As illustrated in FIG. 1, a vacancy 104 can be formed in silicon wafer 102 beneath heater 108 and sensor 110 to help control heat loss between heater 108 and sensor 110.

FIGS. 2A through 2C illustrate an example process to form sensor system 100 as illustrated in FIG. 1. As shown in FIG. 2A, a silicon substrate 102 is provided. Silicon substrate 102 may be processed to include various circuits and other elements outside of the area where sensor system 100 will be formed. As shown in FIG. 2B, vacancy 104 can be formed by etching through silicon substrate 102. This results in a thin layer of silicon, layer 106, on which active components of system 100 are formed. As shown in FIG. 2C, sensor 110 and heater 108 can then be mounted or formed on thin layer 106.

Sensor systems 100 that operate at elevated temperatures consume enormous amounts of energy. Most of this energy goes into powering heater 108 to reach and maintain the elevated temperature. Thermal conductivity of silicon substrate 102, even with a greatly thinned layer 106 on which heater 108 and 110 are mounted, can result in a great deal of energy loss. Further, thermal expansion of thinned layer 106 can result in cracking or other damage to substrate 102.

In order to minimize such energy consumption, the material choice in the device has been re-evaluated. In system 100, silicon substrate 102 has been used due to its familiarity and available processing capabilities. However, use of a silicon substrate is problematic due to its thermal properties.

It is desirable in general that sensor devices use very low electric power to reach the operating temperature. In most sensors developed and manufactured, the MOX sensor is deposited on silicon substrate 110 and heated by a heater 108 that includes an electric coil. The heater coil 108 is placed in the proximity of sensor 110 to allow heat transfer to the sensor through thin layer 106. If the substrate were a solid block, the sensor 110 would only reach the temperature of substrate 102, because of the principles of thermal conduction. The entire substrate 102 would therefore have to be raised to the sensor's operating temperature. In order to overcome this challenge, the thermal mass of the substrate is minimized by thinning only the heated are of the substrate to a few microns—creating a micro-hotplate. As illustrated in FIG. 1, the micro-hotplate 106 can be thinned to around a micron to reduce the thermal mass of substrate 102 in the vicinity of sensor 110. However, when sensor system 100 is mounted on a package substrate or a board, a portion of the heat generated by the heater is dissipated into the package substrate, increasing the demand for electric power needed to reach and maintain the operating temperature.

All present efforts involve using silicon substrates with etched cavities. Some publications indicate filling the cavities in Silicon substrates with synthetic materials that have lower thermal conductivity than air. This is a more expensive manufacturing process. Also, although the conductivity of the synthetic materials is low, the path of thermal conduction through Silicon still exists.

Embodiments of a sensor system according to the present application use a glass substrate instead of a silicon substrate. Use of a glass substrate can improve the power efficiency of the sensor device by minimizing heat dissipated through the substrate. Due to the different thermal conductivity characteristics of glass in comparison with a silicon substrate, using a glass substrate to develop a gas sensor device can greatly decrease the power consumption of the gas sensor system. The power consumption is further decreased by minimizing the heat dissipation into the package substrate, also due to the extremely low thermal conductivity of glass.

Use of glass substrate can be accomplished in multiple ways depending on the design of the sensor system and constraints in manufacturing process flow. In some embodiments, the glass substrate can be thinned by etching a cavity from the backside such that the glass substrate design is similar to that of the silicon substrate illustrated in FIG. 1. In some embodiments, the entire glass substrate can be thinned down (depending on the glass selected and handling capability) creating a thin gas sensor system that can then be mounted on a package without transmitting the heat to the package.

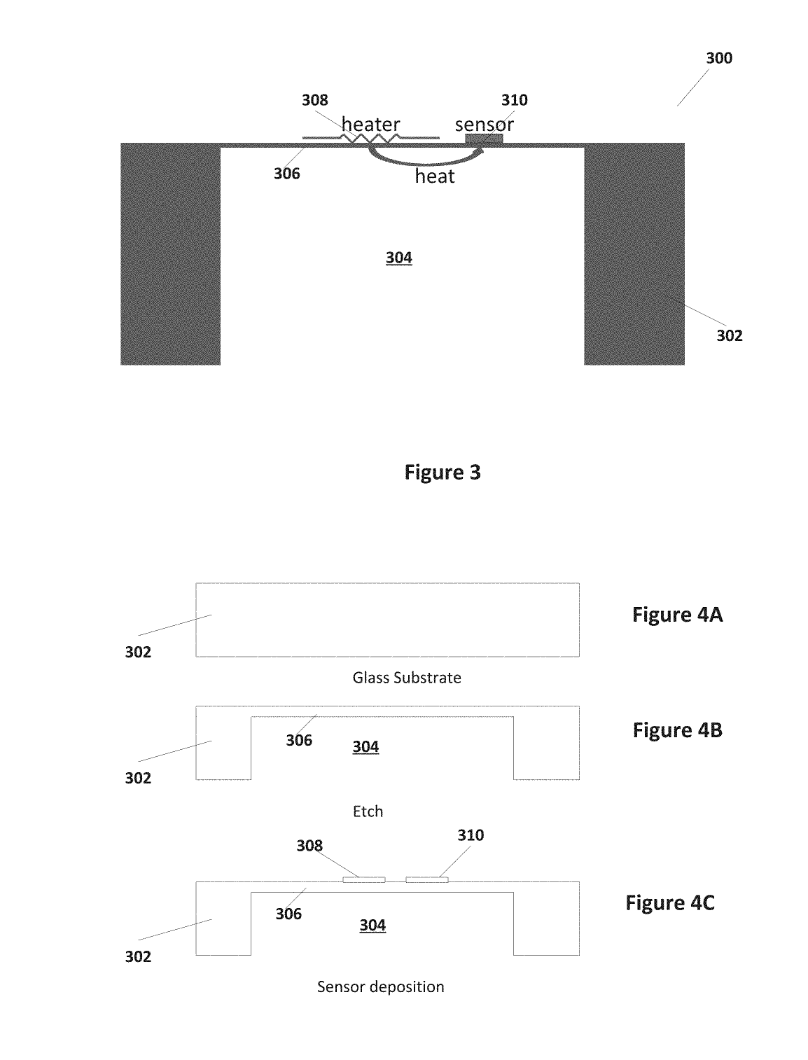

FIG. 3 illustrates a heater system 300 according to some embodiments of the present invention. As illustrated in FIG. 3, heater system 300 is formed on a glass substrate 302. Sensor system 300 includes a cavity 304 where a thin layer 306 is formed in glass substrate 302. A gas sensor 310 and heater 308 are formed on thin layer 306. Because of the low thermal conductivity of glass, the surface glass substrate 302, thin layer 306, that is in contact with heater 308 will reach higher temperatures much quicker than the core of substrate 302. This temperature difference between the surface of thin layer 306 and the core of glass substrate 302 can be very significant and cause enormous stress due to Coefficient of Thermal Expansion (CTE) mismatch, resulting in formation of cracks. The CTE mismatch phenomenon can be thwarted by selection of a glass material for glass substrate 302 that has higher crack resistance and starting with a finely polishing glass substrate 302.

These measures might still not be sufficient if the temperature ramp rate affected by heater 308 is very high. In such cases, thinning of the substrate can greatly increase device reliability. To thin the substrate only in the area of heating, cavity 304 can be chemically etched. Other processes to create cavity 304 such as bead-blasting may also be performed.

FIGS. 4A through 4C illustrate the process of forming gas sensor system 300. As shown in FIG. 4A, a glass substrate 302 can be polished. In FIG. 4B, cavity 304 can be formed by etching a backside of substrate 302. Cavity 304 leaves a thin layer 306, which promotes localization of heating as discussed above. In FIG. 4C, gas sensor 310 and heater 308 are deposited on thin layer 306.

Once cavity 304 is developed in glass substrate 302 in panel or wafer form, glass substrate 302 can be processed like a Silicon wafer for the remainder of the process. If the process flow is such that cavity 304 is etched last, or in the middle of the process, this option may not be applicable considering the interaction of the strong etchants used on glass substrate with the deposited materials of heater 308 and sensor 310. Consequently, formation of cavity 304 can be performed prior to deposition of heater 308 and sensor 310.

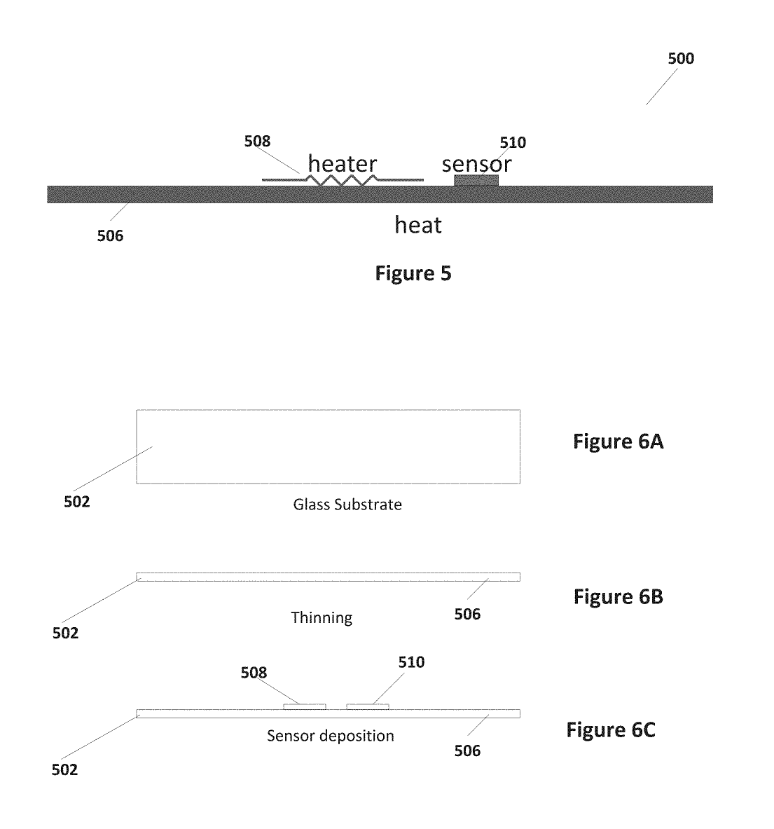

FIG. 5 illustrates another embodiment of gas sensory system according to some embodiments. Gas sensor system 500 is formed on a thin glass substrate 506. As shown in FIG. 5, a heater 508 and a sensor 510 are formed on thin substrate 506.

FIGS. 6A through 6C illustrate a process of forming gas sensor system 500 as illustrated in FIG. 5. As shown in FIG. 6A, a glass substrate 502 is provided. In FIG. 6B, glass substrate 502 is thinned and polished to form thin substrate 502. In FIG. 6C, heater 508 and sensor 510 are provided on substrate 502.

The process illustrated in FIGS. 6A through 6C is suitable if the manufacturing of gas sensor devices involves creating the cavity last, or in the middle of the process flow. The cavity is eliminated by thinning the entire substrate, which can be accomplished using a lapping process. As shown in FIG. 6A, the manufacturing process can start with a wafer or a panel glass substrate 502, which is already thinned or the wafer can be thinned last. The challenge in starting with thinned wafers is wafer handling, unless a temporary carrier wafer is used. The final thickness of the wafers is to be determined based on the lapping process capability, temperature ramp rate supplied by heater 508, resistance of the glass to forming cracks, and other parameters.

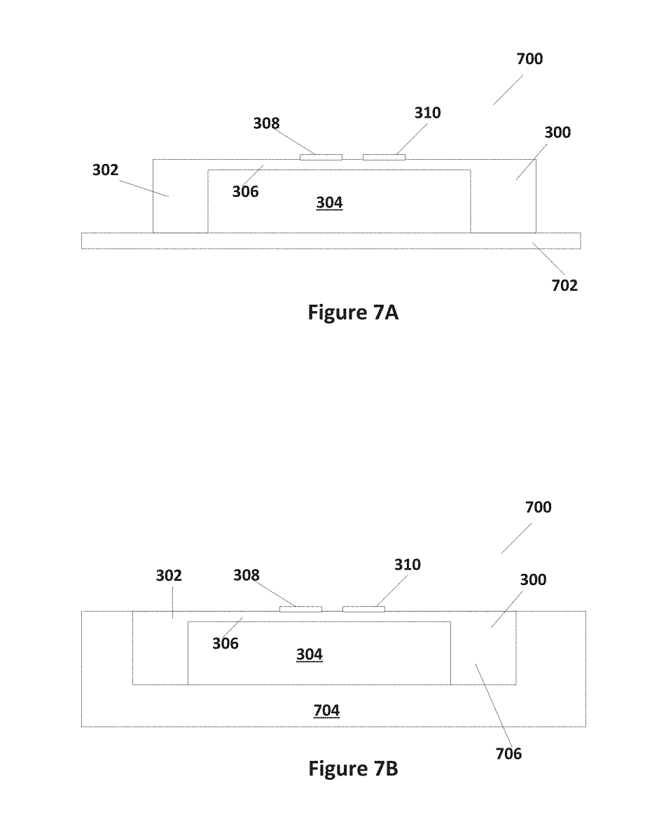

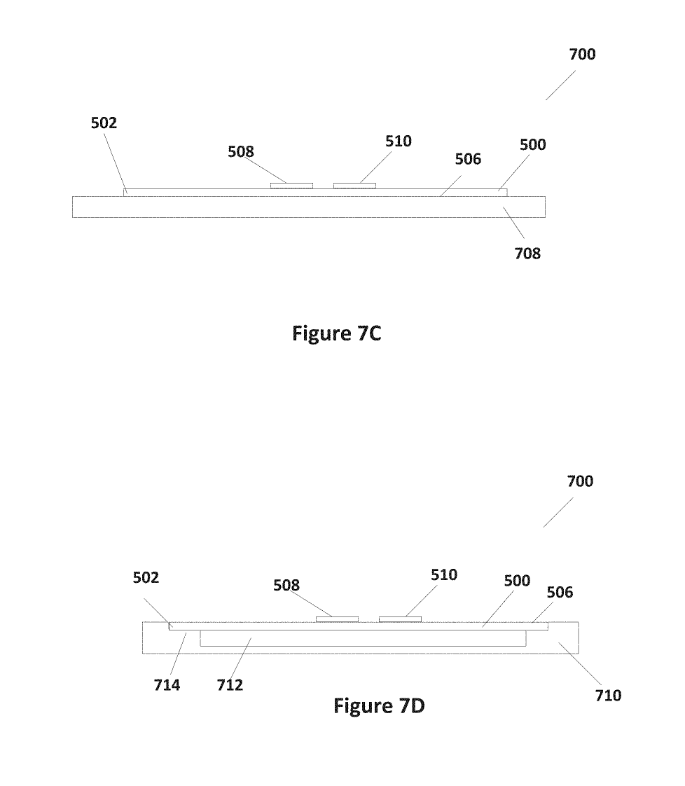

FIGS. 7A through 7D illustrate embodiments of sensor systems according to the present invention embedded into a system 700. FIG. 7A illustrates, for example, sensor system 300 mounted on a substrate 702. Substrate 702 may be a printed circuit board (PCB), a silicon wafer, or other substrate. In some embodiments, circuits that drive heater 308 and interface with sensor 310 are incorporated on substrate 702.

In FIG. 7B, sensor system 300 is embedded in a substrate 704. As shown in FIG. 7B, a cavity 706 large enough to accommodate sensor system 300 is formed in substrate 704 and sensor system 300 is bonded into cavity 706. Substrate 704 may, for example, be a silicon substrate on which circuitry that drives heater 308 and interfaces with sensor 310 are incorporated.

FIG. 7C illustrates sensor system 500 mounted on a substrate 708. As discussed above, substrate 708 may be a PCB or other silicon substrate on which circuitry to drive heater 508 and interface with sensor 510 is provided.

FIG. 7D illustrates sensor system 500 mounted on a substrate 710. Substrate 710 includes a cavity 712 formed in substrate 710 and may include a lip 714 on which sensor system 500 is bonded. Substrate 710 can be a silicon substrate on which circuitry is provided to drive heater 508 and interface with sensor 510.

The above detailed description is provided to illustrate specific embodiments of the present invention and is not intended to be limiting. Numerous variations and modifications within the scope of the present invention are possible. The present invention is set forth in the following claims.

What is claimed is:

1. A system, comprising:
a glass substrate;
a heater formed on the glass substrate; and
a sensor formed adjacent the heater formed on the glass substrate.

2. The system of claim 1, wherein the glass substrate includes a cavity under the heater and the sensor.

3. The system of claim 1, wherein the glass substrate is thin.

4. A method of forming a gas sensor system, comprising:
providing a glass substrate;
forming a heater on the glass substrate; and
forming a sensor adjacent the heater on the glass substrate.

5. The method of claim 4, further including forming a cavity in the glass substrate.

6. The method of claim 4, further including thinning the glass substrate.

7. A system, comprising:
a sensor system having a heater and a sensor formed adjacent to the heater on a glass substrate;
a system substrate on which the sensor system is mounted.

8. The system of claim 7, wherein the system substrate is a printed circuit board.

9. The system of claim 7, wherein the system substrate is a silicon substrate.

10. The system of claim 9, wherein the silicon substrate includes a cavity that receives the sensor system.
